(19) United States
(12) Patent Application Publication
(10) Pub. No.: US 2020/0046245 A1
(43) Pub. Date: Feb. 13, 2020

(54) METHOD AND SYSTEM TO DETECT ATRIAL FLUTTER WAVES IN CARDIAC ACTIVITY SIGNALS

(71) Applicant: Pacesetter, Inc., Sytlmar, CA (US)

(72) Inventors: Fujian Qu, San Jose, CA (US); Gene A. Bornzin, Simi Valley, CA (US); Jong Gill, Valencia, CA (US); Stuart Rosenberg, Castaic, CA (US); Neha Malhotra, Los Angeles, CA (US)

(21) Appl. No.: 16/058,910

(22) Filed: Aug. 8, 2018

(51) Int. Cl. A61B 5/0468; A61B 5/00; A61B 5/0452; A61B 5/0472

(52) U.S. Cl. CPC A61B 5/0468; A61B 5/0006; A61B 5/0472; A61B 5/6861; A61B 5/04525

(57) ABSTRACT

A method and system are provided for detecting arrhythmias in cardiac activity. The method the method and system, under control of one or more processors configured with specific executable instructions, obtain cardiac activity (CA) signals for a series of beats, build a QRS-T template based on an ensemble of QRS complexes within the CA signals, and subtract the QRS-T template from the CA signals to obtain QRS-T scrubbed CA signals. The method and system determine an atrial flutter (AFL) timing feature within the QRS scrubbed CA signals, and declare an AFL episode based on a relation between the AFL timing feature and an AFL cluster criteria.

### FIELD OF THE INVENTION

Embodiments herein relate generally to implantable medical devices, and more particularly to detection and discrimination of atrial flutter waves in cardiac activity signals.

### BACKGROUND OF THE INVENTION

Atrial Flutter (AFL) refers to a set of arrhythmias that originate in re-entrant circuits inside the atria. The rate of AFL is usually from 240 to 340 per minute, with 300 per minute being most often observed. An electro-cardiogram (ECG) presentation of atrial flutter appears as a series of saw tooth or picket fence flutter waves that may exhibit different atrioventricular (AV) conduction ratios, such as 2:1, 3:1, 4:1, or higher, AFL may commonly occur in patients at risk for atrial fibrillation (AF) as well as patients treated with pulmonary vein isolation ablation procedures to treat AF.

Today, commercially released implantable cardiac monitors (ICMs) utilize AF detection algorithms that are tailored to detect atrial fibrillation. However, conventional AF detection algorithms are not well suited to accurately detect AFL. In part, conventional AF detection algorithms have trouble in detecting AFL because ventricular rates associated with AFL may be lower than rates that are programmed for a tachycardia threshold. When an AFL episode occurs at a rate that is lower than a programmed tachycardia threshold, the AFL events are never logged as tachycardia episodes. Also, conventional AF detection algorithms detect AF based on variability in R-wave to R-wave (R-R) intervals over a series of cardiac beats. Patient under AFL rhythm may experience sufficiently regular R-R intervals so that the AF detection algorithm would not declare an arrhythmia detection. In addition, conventional AF detection algorithms are unable to accurately and consistently account for the large morphology variation exhibited by AFL events and small amplitudes of flutter waves relative to R-waves and T-waves.

If AFL episodes could be recognized the arrhythmia may be often readily treated by isthmus ablation. Post RF ablation flutters also are often at a relatively long cycle length and can also be effectively treated by identification of the flutter circuit and ablating across the reentrant path. An opportunity remains to develop automatic AFL detection algorithms for use by implantable cardiac monitor devices.

### SUMMARY

In accordance with embodiments herein, a computer implemented method is provided for detecting arrhythmias in cardiac activity. The method comprises, under control of one or more processors configured with specific executable instructions, obtaining cardiac activity (CA) signals for a series of beats, building a QRS-T template based on an ensemble of QRS complexes within the CA signals, and subtracting the QRS-T template from the CA signals to obtain QRS-T scrubbed CA signals. The method further comprises determining an atrial flutter (AFL) timing feature within the QRS scrubbed CA signals, and declaring an AFL episode based on a relation between the AFL timing feature and an AFL cluster criteria.

Additionally or alternatively, the method further comprises determining cycle lengths of AFL wave features within the QRS scrubbed CA signals, the AFL timing feature determined based on the cycle lengths. The AFL timing feature may represent a distribution of the cycle lengths. The declaring operation may comprise declaring the AFL episode when the AFL timing feature exhibits a distribution of cycle lengths having a select peak within a select timing range. Additionally or alternatively, the method may further comprise, before performing the subtracting operation, aligning the QRS-T template with each QRS-T segment in the CA signals, Additionally or alternatively, the method may further comprise scaling the QRS-T template based on the amplitude of each QRS-T segment in the CA signals before the subtracting operation.

Additionally or alternatively, the building operation may comprise building a QRS-T template and wherein the subtracting operation comprises subtracting the QRS-T template from each QRS-T segment in the CA signals to calculate the QRS-T scrubbed CA signals. Optionally, the QRS-T scrubbed CA signals may include residual QRS complex, the method further comprising applying a blanking mask to blank a QRS segment of the QRS-T scrubbed CA signal to eliminate a residual QRS components from the QRS-T scrubbed CA signals. Optionally, the applying the blanking mask comprises identifying leading and trailing values of the CA signals at the beginning and ending points of the blanking mask and replacing the CA signals with a straight-line signal between the beginning and ending points. Optionally, the method further comprises generating a histogram that maintains a count of AFL cycle lengths within predetermined bins, the determining operation determining whether the count within a select one or more of the predetermined bins exceeds a threshold.

In accordance with embodiments herein, a system is provided for detecting arrhythmias in cardiac activity. The system comprises memory to store specific executable instructions, and one or more processors configured to execute the specific executable instructions for obtaining cardiac activity (CA) signals for a series of beats, and building a QRS-T template based on an ensemble of QRS complexes within the CA signals. The processors are further configured for subtracting the QRS-T template from the CA signals to obtain QRS scrubbed CA signals, determining an atrial flutter (AFL) timing feature within the QRS scrubbed CA signals; and declaring an AFL episode based on a relation between the AFL timing feature and an AFL cluster criteria.

Additionally or alternatively, the one or more processors are further configured to determine cycle lengths of AFL wave features within the QRS scrubbed CA signals, the AFL timing feature determined based on the cycle lengths. The AFL timing feature may represent a distribution of the cycle lengths. Additionally or alternatively, the one or more processors are configured to declare the AFL episode when the AFL timing feature exhibits a distribution of cycle lengths having a select peak within a select timing range. Additionally or alternatively, the one or more processors are further configured to align the QRS-T template with an R-wave marker of a QRS complex of the CA signals before performing the subtracting operation.

Additionally or alternatively, the one or more processors are further configured to scale the QRS-Template based on the amplitude of QRS-T segment in the CA signals before the subtracting operation. The building operation may comprise building, as the QRS-Template, a QRS-T template and wherein the subtracting operation comprises subtracting the QRS-T template from the CA signals to calculate the QRS-T scrubbed CA signals. The QRS scrubbed CA signals may include residual QRS complex, and wherein the one or more processors are configured to apply a blanking mask to blank a QRS segment of the QRS-T scrubbed CA signal to eliminate a residual QRS components from the QRS-T scrubbed CA signals. Additionally or alternatively, the system may further comprise an implantable medical device housing the processor and memory. Additionally or alternatively, the processor and memory may be housed within at least one of a local external device and a remote server.

### BRIEF DESCRIPTION OF THE DRAWINGS

Figure illustrates an implantable cardiac monitoring device (ICM) intended for subcutaneous implantation at a site near the heart in accordance with embodiments herein.

FIG. 2 shows a block diagram of the ICM formed in accordance with embodiments herein.

FIG. 3A illustrates a process for analyzing CA signals in the time domain and detecting atrial flutter episodes in accordance with embodiments herein.

FIG. 3B illustrates an example of CA signals that are collected while a patient is experiencing atrial flutter.

FIG. 3C illustrates a graphical representation of the subtraction operation for a QRS-T segment.

FIG. 3D illustrates a graphical representation of the masking operation for a QRS-T segment.

FIG. 3E illustrates a blanked and QRS-T scrubbed CA signal segment analyzed in accordance with the embodiments herein.

FIG. 3F illustrates a graphical representation of an AFL distribution of AFL cycle lengths determined in accordance with an embodiment herein.

FIG. 4 illustrates a process for building a QRS-T template in accordance with the embodiments herein.

### DETAILED DESCRIPTION

The terms “cardiac activity signal”, “cardiac activity signals”, “CA signal” and “CA signals” (collectively “CA signals”) are used interchangeably throughout to refer to an analog or digital electrical signal recorded by two or more electrodes positioned subcutaneous or cutaneous, where the electrical signals are indicative of cardiac electrical activity. The cardiac activity may be normal/healthy or abnormal/arrhythmic. Non-limiting examples of CA signals include ECG signals collected by cutaneous electrodes, and EGM signals collected by subcutaneous electrodes.

The terms “cardiac activity data set” and “CA data set” (collectively “CA data set”) are used interchangeably to refer to a data set that includes measured CA signals for a series of cardiac events in combination with device documented markers.

The term “marker” refers to data and/or information identified from CA signals that may be presented as graphical and/or numeric indicia indicative of one or more features within the CA signals and/or indicative of one or more episodes exhibited by the cardiac events. Markers may be superimposed upon CA signals or presented proximate to, and temporally aligned with, CA signals. Non-limiting examples of markers may include R-wave markers, noise markers, activity markers, interval markers, refractory markers, P-wave markers, T-wave markers, PVC markers, sinus rhythm markers, AF markers and other arrhythmia markers. As a further nonlimiting example, basic event markers may include “AF entry” to indicate a beginning of an AF event, “in AF” to indicate that AF is ongoing, “AF exit” to indicate that AF has terminated, “T” to indicate a tachycardia beat, “B” to indicate a bradycardia heat, “A” to indicate an asystole beat, “VS” to indicate a regular sinus beat, “Tachy” to indicate a tachycardia episode, “Brady” to indicate a Bradycardia episode, “Asystole” to indicate an asystole episode, “Patient activated” to indicate a patient activated episode. An activity marker may indicate activity detected by activity sensor during the CA signal Noise markers may indicate entry/start, ongoing, recovery and exit/stop of noise. Markers may be presented as symbols, dashed lines, numeric values, thickened portions of a waveform, and the like. Markers may represent events, intervals, refractory periods, ICM activity, and other algorithm related activity. For example, interval markers, such as the R-R interval, may include a numeric value indicating the duration of the interval. The AF markers indicate atrial fibrillation rhythmic.

The term “device documented marker” refers to markers that are declared by an implantable cardiac monitor and/or implantable medical device. Any or all of the foregoing examples of markers represent device document markers. Markers may be declared based on numerous criteria, such as signal processing, feature detection and AF detection software and hardware within and/or operating on the implantable cardiac monitor and/or implantable medical device.

The term “FOI” refers to a feature of interest within CA signals, Nonlimiting examples of features of interest include an R-wave, P-wave, T-wave and isoelectric segments. A feature of interest may correspond to a peak of an individual R-wave, an average or median P, R or T-wave peak and the like.

The terms “beat” and “cardiac event” are used interchangeably and refer to both normal or abnormal events.

The terms “normal” and “sinus” are used to refer to events, features, and characteristics of, or appropriate to, a heart's healthy or normal functioning.

The terms “abnormal” or “arrhythmic” are used to refer to events, features, and characteristics of, or appropriate to, a un-healthy or abnormal functioning of the heart.

The term “real-time” refers to a time frame contemporaneous with normal or abnormal episode occurrences. For example, a real-time process or operation would occur during or immediately after (e.g., within minutes or seconds after) a cardiac event, a series of cardiac events, an arrhythmia episode, and the like.

FIG. 1 illustrates an implantable cardiac monitoring device (ICM) 100 intended for subcutaneous implantation at a site near the heart. The ICM 100 includes a pair of spaced-apart sense electrodes 114, 126 positioned with respect to a housing 102. The sense electrodes 114, 126 provide for detection of far field electrogram signals. Numerous configurations of electrode arrangements are possible. For example, the electrode 114 may be located on a distal end of the ICM 100, while the electrode 126 is located on a proximal side of the ICM 100. Additionally or alternatively, electrodes 126 may be located on opposite sides of the ICM 100, opposite ends or elsewhere. The distal electrode 114 may be formed as part of the housing 102, for example, by coating all but a portion of the housing with a nonconductive material such that the uncoated portion forms the electrode 114, In this case, the electrode 126 may be electrically isolated from the housing 102 electrode by placing it on a component separate from the housing 102, such as the header 120. Optionally, the header 120 may be formed as an integral portion of the housing 102, The header 120 includes an antenna 128 and the electrode 126. The antenna 128 is configured to wirelessly communicate with an external device 154 in accordance with one or more predetermined wireless protocols (e.g., Bluetooth, Bluetooth low energy, Wi-Fi, etc.). The housing 102 includes various other components such as: sense electronics for receiving signals from the electrodes, a microprocessor for processing the signals in accordance with algorithms, such as the AF detection algorithm described herein, a loop memory for temporary storage of CA data, a device memory for long-term storage of CA data upon certain triggering events, such as AF detection, sensors for detecting patient activity and a battery for powering components.

In at least some embodiments, the ICM 100 is configured to be placed subcutaneously utilizing a minimally invasive approach. Subcutaneous electrodes are provided on the housing 102 to simplify the implant procedure and eliminate a need for a transvenous lead system. The sensing electrodes may be located on opposite sides of the device and designed to provide robust episode detection through consistent contact at a sensor-tissue interface. The ICM 100 may be configured to be activated by the patient or automatically activated, in connection with recording subcutaneous ECG signals.

The ICM 100 senses far field, subcutaneous CA signals, processes the CA signals to detect arrhythmias and if an arrhythmia is detected, automatically records the CA signals in memory for subsequent transmission to an external device. The CA signal processing and AF detection is provided for, at least in part, by algorithms embodied in or implemented by the microprocessor. The ICM 100 includes one or more processors and memory that stores program instructions directing the processors to implement AF detection utilizing an on-board R-R interval irregularity (ORI) process that analyzes cardiac activity signals collected over one or more sensing channels.

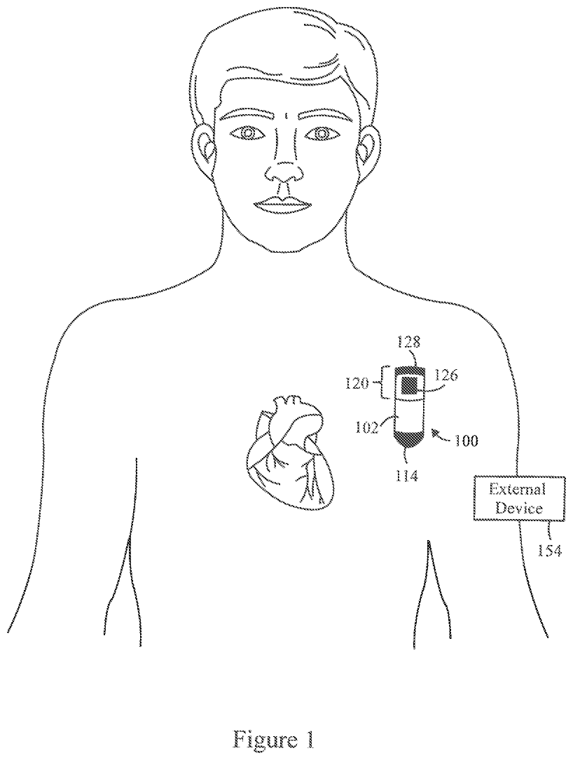

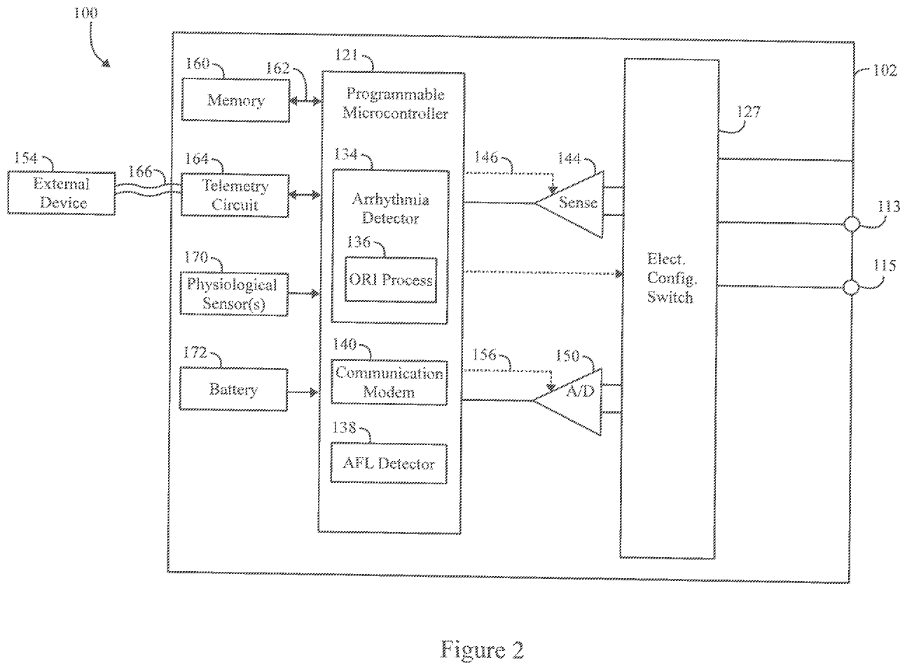

FIG. 2 shows a block diagram of the ICM 100 formed in accordance with embodiments herein. The ICM 100 may be implemented to monitor ventricular activity alone, or both ventricular and atrial activity through sensing circuit. The ICM 100 has a housing 102 to hold the electronic/computing components. The housing 102 (which is often referred to as the “can”, “case”, “encasing”, or “case electrode”) may be programmably selected to act as an electrode for certain sensing modes. Housing 102 further includes a connector (not shown) with at least one terminal 113 and optionally additional terminals 115. The terminals 113, 115 may be coupled to sensing electrodes that are provided upon or immediately adjacent the housing 102. Optionally, more than two terminals 113, 115 may be provided in order to support more than two sensing electrodes, such as for a bipolar sensing scheme that uses the housing 102 as a reference electrode. Additionally or alternatively, the terminals 113, 115 may be connected to one or more leads having one or more electrodes provided thereon, where the electrodes are located in various locations about the heart. The type and location of each electrode may vary.

The ICM 100 includes a programmable microcontroller 121 that controls various operations of the ICM 100, including cardiac monitoring. Microcontroller 121 includes a microprocessor (or equivalent control circuitry), RAM and/or ROM memory, logic and timing circuitry, state machine circuitry, and I/O circuitry. The microcontroller 121 also performs the operations described herein in connection with collecting cardiac activity data and analyzing the cardiac activity data.

A switch 127 is optionally provided to allow selection of different electrode configurations under the control of the microcontroller 121. The electrode configuration switch 127 may include multiple switches for connecting the desired electrodes to the appropriate I/O circuits, thereby facilitating electrode programmability. The switch 127 is controlled by a control signal from the microcontroller 121. Optionally, the switch 127 may be omitted and the I/O circuits directly connected to the housing electrode 114 and a second electrode 126.

Microcontroller 121 includes an arrhythmia detector 134 that is configured to analyze cardiac activity signals to identify potential AF episodes as well as other arrhythmias (e.g., Tachcardias, Bradycardias, Asystole, etc.). By way of example, the arrhythmia detector 134 may implement an AF detection algorithm as described in U.S. Pat. No. 8,135,456, the complete subject matter of which is incorporated herein by reference. Although not shown, the microcontroller 121 may further include other dedicated circuitry and/or firmware/software components that assist in monitoring various conditions of the patient's heart and managing pacing therapies. The arrhythmia detector 134 of the microcontroller 121 includes an on-board R-R interval irregularity (ORI) process 136 that detects AF episodes using R-R interval irregularities. The ORI process 136 may be implemented as firmware, software and/or circuits. The ORI process 136 uses a hidden Markov Chains and Euclidian distance calculations of similarity to assess the transitionary behavior of one R-wave (RR) interval to another and compare the patient's RR interval transitions to the known RR interval transitions during AF and non-AF episodes obtained from the same patient and/or many patients.

The microcontroller 121 also includes an AFL detector 138 that is configured to implement the operations described herein. Among other things, the AFL detector 138 is configured for obtaining far field cardiac activity (CA) signals for a series of beats; building a QRS-Template based on an ensemble of QRS complexes within the CA signals; subtracting the QRS-Template from the CA signals to obtain QRS-T scrubbed CA signals; determining an atrial flutter (AFL) timing feature within the QRS-T scrubbed CA signals; and declaring an AFL episode based on a relation between the AFL timing feature and an AFL cluster criteria. The AFL detector 138 may be configured to identify AFL wave features within the corresponding beats of the QRS-T scrubbed CA signals. The AFL timing feature may represent at least one of cycle lengths between the AFL wave features and a distribution of the AFL wave features. The AFL detector 138 may declare an AFL episode when the AFL timing feature for a select number of beats fall within an AFL cluster range. The AFL detector 138 may perform the subtracting operation in connection with each beat of the CA signals, and align an R-wave feature of the QRS-Template with an R-wave marker of a QRS complex of the CA signals.

The AFL detector 138 is further configured to determine cycle lengths of AFL wave features within the QRS scrubbed CA signals, the AFL timing feature determined based on the cycle lengths. The AFL timing feature represents a distribution of the cycle lengths. The AFL detector 138 is configured to declare the AFL episode when the AFL timing feature exhibits a distribution of cycle lengths having a select peak within a select timing range. The AFL detector 138 is further configured to align an R-wave feature of the QRS-Template with an R-wave marker of a QRS complex of the CA signals before performing the subtracting operation. The AFL detector 138 is further configured to scale the QRS-Template based on a dynamic range of the QRS scrubbed CA signals before the subtracting operation. The AFL detector 138 is further configured to build, as the QRS-Template, a QRS-T template and wherein the subtracting operation comprises subtracting the QRS-T template from the CA signals such that the QRS scrubbed CA signals represents QRS-T scrubbed CA signals. The AFL detector 138 is configured to apply a blanking mask to the QRS scrubbed CA signal to blank a QRS segment of the QRS-T scrubbed CA signal to remove/eliminate a residual QRS components from the QRS-T scrubbed CA signals.

The ICM 100 is further equipped with a communication modem (modulator/demodulator) 140 to enable wireless communication. In one implementation, the communication modem 140 uses high frequency modulation, for example using RE, Bluetooth or Bluetooth Low Energy telemetry protocols. The signals are transmitted in a high frequency range and will travel through the body tissue in fluids without stimulating the heart or being felt by the patient. The communication modem 140 may be implemented in hardware as part of the microcontroller 121, or as software/firmware instructions programmed into and executed by the microcontroller 121. Alternatively, the modem 140 may reside separately from the microcontroller as a standalone component. The modem 140 facilitates data retrieval from a remote monitoring network. The modem 140 enables timely and accurate data transfer directly from the patient to an electronic device utilized by a physician.

The ICM 100 includes sensing circuit 144 selectively coupled to one or more electrodes that perform sensing operations, through the switch 127 to detect cardiac activity data indicative of cardiac activity. The sensing circuit 144 may include dedicated sense amplifiers, multiplexed amplifiers, or shared amplifiers. It may further employ one or more low power, precision amplifiers with programmable gain and/or automatic gain control, bandpass filtering, and threshold detection circuit to selectively sense the features of interest. In one embodiment, switch 127 may be used to determine the sensing polarity of the cardiac signal by selectively closing the appropriate switches.

The output of the sensing circuit 144 is connected to the microcontroller 121 which, in turn, determines when to store the cardiac activity data of CA signals (digitized by the A/D data acquisition system 150) in the memory 160. For example, the microcontroller 121 may only store the cardiac activity data (from the ND data acquisition system 150) in the memory 160 when a potential AF episode is detected. The sensing circuit 144 receives a control signal 146 from the microcontroller 121 for purposes of controlling the gain, threshold, polarization charge removal circuitry (not shown), and the timing of any blocking circuitry (not shown) coupled to the inputs of the sensing circuit.

Optionally, the ICM 100 may include multiple sensing circuits, similar to sensing circuit 144, where each sensing circuit is coupled to two or more electrodes and controlled by the microcontroller 121 to sense electrical activity detected at the corresponding two or more electrodes. The sensing circuit 144 may operate in a unipolar sensing configuration or in a bipolar sensing configuration. Optionally, the sensing circuit 144 may be removed entirely and the microcontroller 121 perform the operations described herein based upon the CA signals from the ND data acquisition system 150 directly coupled to the electrodes.

The ICM 100 further includes an analog-to-digital A/D data acquisition system (DAS) 150 coupled to one or more electrodes via the switch 127 to sample cardiac activity signals across any pair of desired electrodes. The data acquisition system 150 is configured to acquire cardiac electrogram (EGM) signals as CA signals, convert the raw analog data into digital data, and store the digital data as CA data for later processing and/or telemetric transmission to an external device 154 (e.g., a programmer, local transceiver, or a diagnostic system analyzer). The data acquisition system 150 is controlled by a control signal 156 from the microcontroller 121. The EGM signals may be utilized as the cardiac activity data that is analyzed for potential AF episodes. The ACS adjustment and ORI process 136 may be applied to signals from the sensing circuit 144 and/or the DAS 150.

By way of example, the external device 154 may represent a bedside monitor installed in a patient's home and utilized to communicate with the ICM 100 while the patient is at home, in bed or asleep. The external device 154 may be a programmer used in the clinic to interrogate the ICM 100, retrieve data and program detection criteria and other features. The external device 154 may be a handheld device (e.g., smartphone, tablet device, laptop computer, smartwatch and the like) that can be coupled over a network (e.g., the Internet) to a remote monitoring service, medical network and the like. The external device 154 facilitates access by physicians to patient data as well as permitting the physician to review real-time CA signals while collected by the ICM 100.

The microcontroller 121 is coupled to a memory 160 by a suitable data/address bus 162. The programmable operating parameters used by the microcontroller 121 are stored in memory 160 and used to customize the operation of the ICM 100 to suit the needs of a particular patient. Such operating parameters define, for example, detection rate thresholds, sensitivity, automatic features, AF detection criteria, activity sensing or other physiological sensors, and electrode polarity, etc.

In addition, the memory 160 stores the cardiac activity data, as well as the markers and other data content associated with detection of arrhythmia episodes. The operating parameters of the ICM 100 may be non-invasively programmed into the memory 160 through a telemetry circuit 164 in telemetric communication via communication link 166 with the external device 154. The telemetry circuit 164 allows intracardiac electrograms and status information relating to the operation of the ICM 100 (as contained in the microcontroller 121 or memory 160) to be sent to the external device 154 through the established communication link 166. In accordance with embodiments herein, the telemetry circuit 164 conveys the cardiac activity data, markers and other information related to AF episodes.

The ICM 100 may further include magnet detection circuitry (not shown), coupled to the microcontroller 121, to detect when a magnet is placed over the unit. A magnet may be used by a clinician to perform various test functions of the housing 102 and/or to signal the microcontroller 121 that the external device 154 is in place to receive or transmit data to the microcontroller 121 through the telemetry circuits 164.

The ICM 100 can further include one or more physiologic sensors 170. Such sensors are commonly referred to (in the pacemaker arts) as “rate-responsive” or “exercise” sensors. The physiological sensor 170 may further be used to detect changes in the physiological condition of the heart, or diurnal changes in activity (e.g., detecting sleep and wake states). Signals generated by the physiological sensors 170 are passed to the microcontroller 121 for analysis and optional storage in the memory 160 in connection with the cardiac activity data, markers, episode information and the like. While shown as being included within the housing 102, the physiologic sensor(s) 170 may be external to the housing 102, yet still be implanted within or carried by the patient, Examples of physiologic sensors might include sensors that, for example, activity, temperature, sense respiration rate, pH of blood, ventricular gradient, activity, position/posture, minute ventilation (MV), and so forth.

A battery 172 provides operating power to all of the components in the ICM 100, The battery 172 is capable of operating at low current drains for long periods of time. The battery 172 also desirably has a predictable discharge characteristic so that elective replacement time can be detected. As one example, the housing 102 employs lithium/silver vanadium oxide batteries. The battery 172 may afford various periods of longevity (e.g., three years or more of device monitoring). In alternate embodiments, the battery 172 could be rechargeable. See for example, U.S. Pat. No. 7,294,108, Cardiac event micro-recorder and method for implanting same, which is hereby incorporated by reference.

The ICM 100 provides a simple to configure data storage option to enable physicians to prioritize data based on individual patient conditions, to capture significant events and reduce risk that unexpected events are missed. The ICM 100 may be programmable for pre- and post-trigger event storage. For example, the ICM 100 may be automatically activated to store 10-120 seconds of CA data prior to an event of interest and/or to store 10-120 seconds of post CA data. Optionally, the ICM 100 may afford patient triggered activation in which pre-event CA data is stored, as well as post event CA data (e.g., pre-event storage of 1-15 minutes and post-event storage of 1-15 minutes), Optionally, the ICM 100 may afford manual (patient triggered) or automatic activation for CA data. Optionally, the ICM 100 may afford additional programming options (e.g., asystole duration, bradycardia rate, tachycardia rate, tachycardia cycle count). The amount of CA data storage may vary based upon the size of the memory 160.

The ICM 100 may provide comprehensive safe diagnostic data reports including a summary of heart rate, in order to assist physicians in diagnosis and treatment of patient conditions. By way of example, reports may include episodal diagnostics for auto trigger events, episode duration, episode count, episode date/time stamp and heart rate histograms. The ICM 100 may be configured to be relatively small (e.g., between 2-10 cc in volume) which may, among other things, reduce risk of infection during implant procedure, afford the use of a small incision, afford the use of a smaller subcutaneous pocket and the like. The small footprint may also reduce implant time and introduce less change in body image for patients.

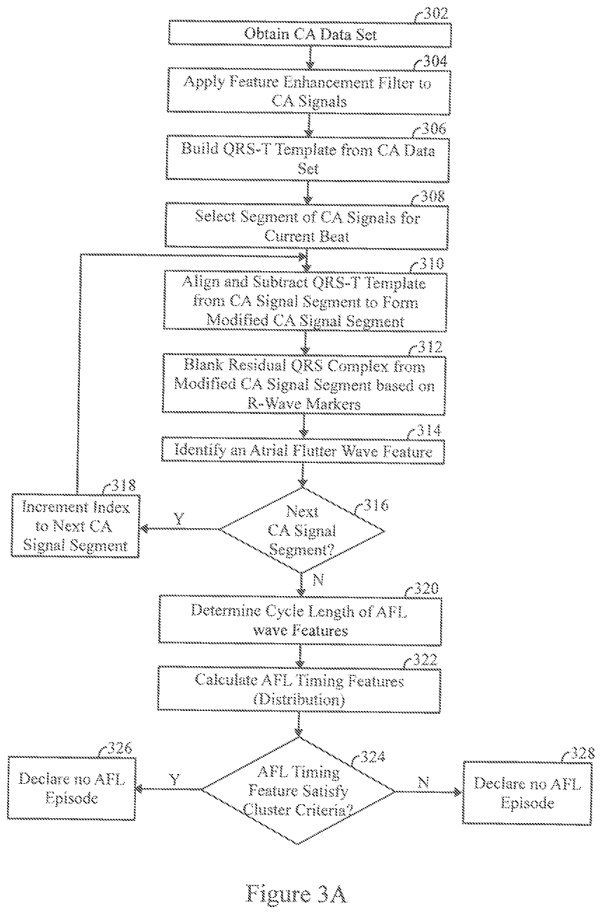

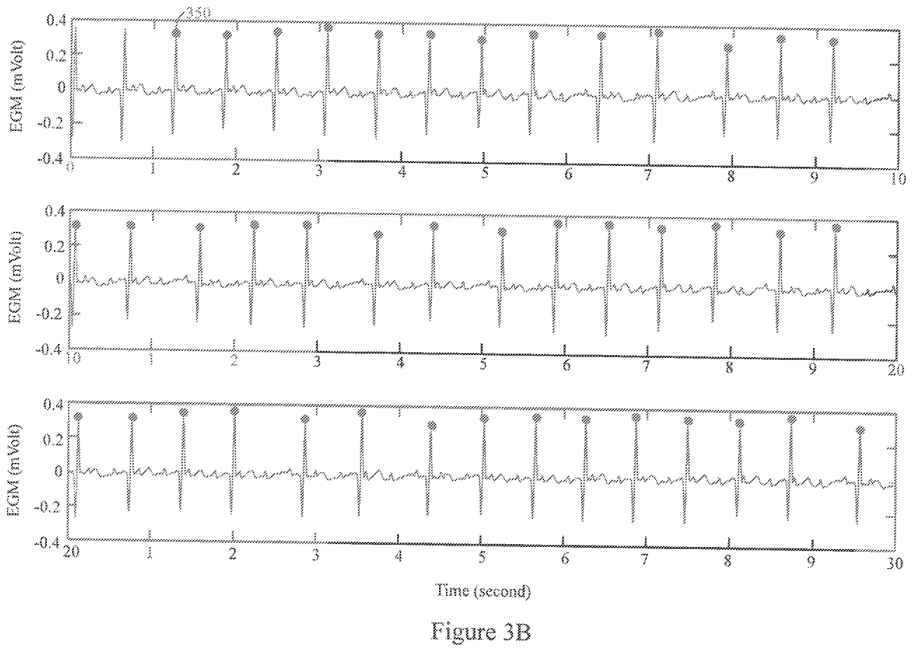

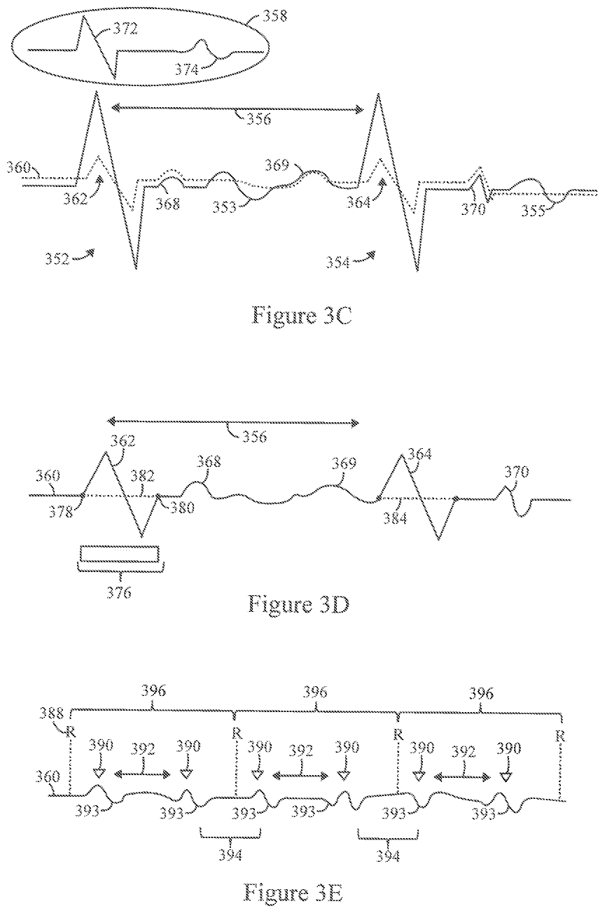

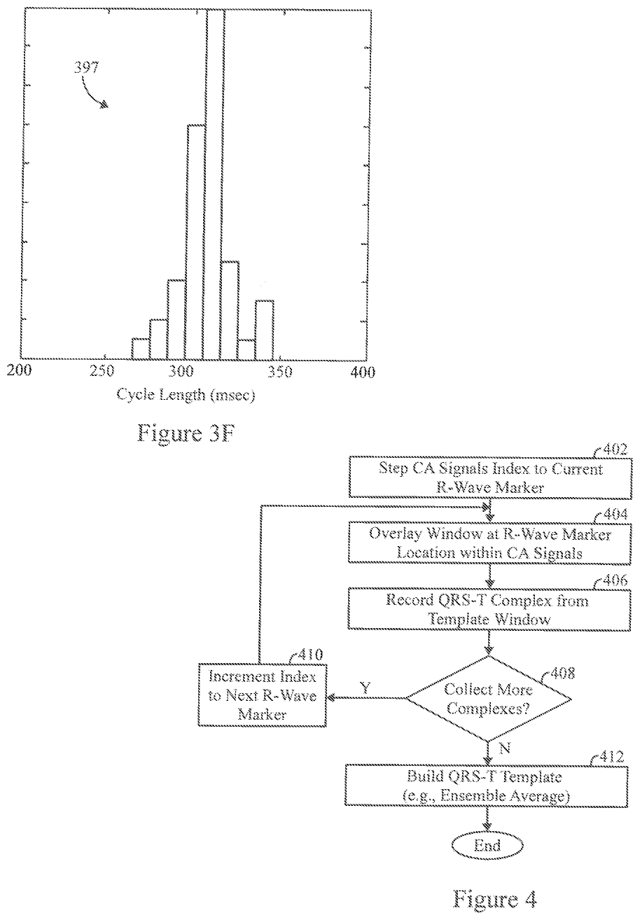

FIG. 3A illustrates a process for analyzing CA signals in the time domain and detecting atrial flutter episodes in accordance with embodiments herein. At 302, one or more processors of the system obtain a cardiac activity (CA) data set including CA signals recorded in connection with a series of cardiac events. The CA data includes device documented rhythmic markers (e.g., R-wave markers) that identify the cardiac beats sensed by the device within the series of cardiac events. For example, the device documented markers may be declared and designated by the ICM utilizing an OR process to analyze the CA signals. ECG and/or EGM signals may be collected by a subcutaneous ICM that does not include a transvenous lead. The cardiac activity data may have been previously acquired and stored in memory of an implantable or external monitoring device, implantable or external therapy delivery device, programmer, workstation, healthcare network or other system. When the cardiac activity data has been previously acquired, the obtaining operation at 302 represents accessing and reading the previously stored cardiac activity data.

The operations of FIG. 3A may be staged to be performed upon the CA data at various times, such as in real time (e.g., during or shortly after a patient experiences an episode) or at any time after storage of the CA data. The operations of FIG. 3A may be performed by devices and systems at various proximity to a patient with the ICM. For example, the CA data may be read out of an ICM and transmitted to a local portable external device (e.g., smartphone, table computer, laptop computer, smartwatch, etc.), where the local portable external device locally implements all or a portion of the operations described in connection with FIG. 3A while in close proximity to the patient. Additionally or alternatively, the CA data may be read out of the ICM to a local portable external device and transmitted to a remote server, medical network, physician computer and the like, which implements all or a portion of the operations described in connection with FIG. 3A remote from the patient. Additionally or alternatively, the CA data may be read from the ICM by a programmer device, such as during a patient visit to a physician, where the programmer device implements all or a portion of the operations described in connection with FIG. 3A during or after a patient-doctor visit. The process of FIG. 3A may be implemented as part of a first pass process, in which AFL episodes are identified during or before declaration of another arrhythmia episode (e.g., AF). Additionally or alternatively, the process of FIG. 3A may be implemented as part of a second pass confirmatory process, in which AFL episodes are identified within a CA data set that has already been analyzed and declared to include one or more arrhythmia episodes (e.g., AFL, AF).

The CA data may include CA signals for a series of cardiac events spanning over various periods of time. As one example one segment or set of the cardiac activity data may be collected for an interval that is 30 seconds to 5 minutes in length and that includes one or more ICM declared AF episodes. As another example, one segment or set of the cardiac activity data may be collected for an interval that begins 10-60 seconds before an episode of interest (e.g., an AF episode) and that ends 10-60 seconds after the episode of interest, A CA data set may include one or multiple AF episodes. The duration of a CA data set may be programmed for a predetermined period of time based on detection of AF episodes and/or based on other criteria. The predetermined period of time may be programmed by a clinician, or automatically updated by one or more processors throughout operation, By way of example, the predetermined period of time may correspond to one minute, 30 minutes, one hour or otherwise. The CA data obtained at 302 may correspond to one detected AF episode and/or multiple detected AF episodes. The CA data set obtained at 302 may correspond to one continuous series of cardiac events (e.g., 1 continuous series for 30 seconds to 5 minutes) and/or separate sets of cardiac events (3-10 separate series, each for 30 seconds to 3 minutes of cardiac events).

Collection and analysis of CA signals by the ICM may be initiated automatically when the ICM detects an episode of interest. Additionally or alternatively, the ICM may collect and analyze CA signals in response to a user-initiated instruction. For example, a user may utilize a smart phone or other portable device to establish a communications session with the ICM and instruct the ICM to begin to collect and analyze cardiac signals, such as when the patient is experiencing discomfort, feeling faint, a rapid heart rate, etc.

FIG. 3B illustrates an example of CA signals (e.g., a 30 second strip of EGM signals) that are collected while a patient is experiencing atrial flutter. Circular points 350 indicate device documented R-wave markers determined by the ICM while analyzing the CA signals. Intermediate segments between the R-waves exhibit a flutter wave that is visible but exhibits a relatively small amplitude as compared to the amplitude of the R-waves. The amplitude of the flutter waves is also smaller than amplitude of T-waves within the CA signals. The flutter waves can occur at the same time of T-waves and still visible in FIG. 3B.

Returning to FIG. 3A, at 304, optionally, the one or more processors apply a feature enhancement filter to the CA signals to form an enhanced CA signals in which sinus features of interest are enlarged or exaggerated relative to the original/baseline CA signals. Additionally or alternatively, the processors may up-sample the CA signals from a first sample rate to a second higher sample rate (e.g., from 128 Hz to 512 Hz using Shannon interpolation). Up-sampling the CA signals allows for more precise estimation of the QRS-T templates and finer resolution on the timing for CA signal alignment, Optionally, the operation at 304 may be omitted entirely

At 306, the one or more processors build a QRS-T template from the CA signals. An example process for building a QRS-T template is described in connection with FIG. 4. As explained below in connection with FIG. 4, the QRS-T template is built by combining segments of the CA signals surrounding device documented R-wave markers. For example, a segment beginning 300 ms before and continuing 400 ms after each R-wave marker may be collected from the detected beats. The collection of segments may be combined, such as through an ensemble average. The QRS-T template will exhibit a morphology that generally corresponds to an R-wave and a T-wave for the patient, but will not exhibit a particular morphology for a flutter wave. The ensemble average does not include a flutter wave component as flutter waves occur at different times in a cardiac cycle and thus are generally not temporally aligned with the R-wave (or a device documented R-wave marker). Accordingly, the ensemble averaging process tends to cancel out the flutter wave components.

Optionally, the QRS-T template may be obtained for various heart rate ranges. For example, one QRS-T template may be for the heart rate of 80-100, while another QRS-T template may be for the heart rate of 100-120, etc. Rate sensitive QRS-T templates may be used to account for varying QR-T intervals with respect to heart rate. Rate specific QRS-T templates may be used during a ‘subtraction’ operation described below.

At 308, the one or more processors select a current QRS-T segment of the CA signals for a current beat/cardiac event.

At 310, the one or more processors align the QRS (or QRS-T) template with the current QRS-T segment of the CA signals. The one or more processors subtract the QRS (or QRS-T) template from the QRS-T segment of each detected beat in the CA signals to calculate a QRS-T scrubbed CA signal. As noted herein, QRS-T templates may be obtained for various heart rate ranges, to account for varying QR-T intervals with respect to heart rate. The rate specific QRS-T templates may be used during the subtraction operation. Before performing the subtraction operation, the processors achieve a desired level of alignment between the QRS-T template and the current QRS-T segment of the CA signals. For example, the alignment may be achieved based on R wave peak timing or cross-correlation between the template, and current QRS-T segment. For example, the processors may perform a correlation comparison between the QRS-T template and current segment and identify a point where a local correlation coefficient is at a maximum between the QRS-T template and the current QRS-T segment.

Optionally, before performing the subtraction operation, the QRS-T template may be “scaled” to account for amplitude variations between the QRS-T template and current QRS-T segment. For example, amplitudes of R-waves and T-waves may vary as much as 10% or more from beat to beat. Embodiments herein account for amplitude variations by increasing or decreasing a dynamic range (e.g., peak to peak variation) of the QRS-T template to generally correspond to a dynamic range (e.g., peak to peak variation) of a current QRS-T segment of the CA signals. For example, prior to subtraction, the amplitude of the R and T-wave in the QRS-T template can be adjusted to match the current QRS-T segment by multiplying the QRS-T template by a gain factor, G, at the point of maximum correlation, T. The gain factor minimizes the least squared error and the mathematical formula for the gain is: G=[ΣE(n)*EGM(n−T)]/[E(n)*E(n)], where E(n) represents the QRS-T template and EGM(n) corresponds to an electrogram cardiac activity signal (as the current QRS-T segment of the CA signal) at the time of the greatest correlation.

The alignment, scaling and subtraction operations may not entirely remove the QRS-T complex from the current QRS-T segment of the CA signals and thus, the QRS-T scrubbed CA signal segment may include a residual QRS complex and/or T complex.

FIG. 3C illustrates a graphical representation of the subtraction operation for a QRS-T segment. FIG. 3C illustrates a QRS-T segment (in solid lines) of the CA signals that includes two R-waves 352, 354 separated by an RR interval 356, T-waves 353, 355, and AFL waves 368-370. A QRS-T template 358 is also illustrated and includes an R-wave template 372 and a T-wave template 374. The QRS-T template 358 can be optionally scaled (e.g., the dynamic range is adjusted) and aligned with each of the R-waves 352 and 354, and T-waves 353, 355, and subtracted therefrom to calculate a QRS-T scrubbed CA signal segment 360 (shown in dashed lines). The QRS-T scrubbed CA signal segment 360 includes AFL waves 368-370 and residual R-waves 362, 364, whereas a contribution of the T-waves 353, 355 is substantially or entirely removed (as denoted by the generally straight dashed line through the T-waves 353, 355).

Returning to FIG. 3A, at 312, the one or more processors apply a blanking mask to blank a QRS segment of the QRS-T scrubbed CA signal to remove/eliminate a residual QRS components from the QRS-T scrubbed CA signals. The T-wave is not blanked to avoid impacting accuracy of AFL detection because the AFL wave may occur on top of the T-wave. The processors align the blanking mask with the QRS-T scrubbed CA signal segment based on a position of a corresponding R-wave marker associated with the current segment of the CA signals. The R-wave marker is a device documented marker declared by the ICM and stored with the original CA signals in the CA data set. By way of example, the blanking mask may be centered at the R-wave marker or R-wave peak and set to extend a predetermined distance/time before and after the R-wave marker. The CA signals within the blanking mask (e.g., a 60-75 ms window around the R-wave) are replaced by a linear interpolation signal that extends between and is based on the boundary values of the CA signals at the left and right ends of the blanking mask. For example, the processors may identify leading and trailing values of the CA signals at a beginning and an end of the blanking mask. The processors draw a straight line signal between the leading and trailing values of the CA signals and replace the CA signal with the straight line signal in the portion of the QRS-T scrubbed CA signal aligned with the blanking mask.

FIG. 3D illustrates a graphical representation of the masking operation for a QRS-T segment. FIG. 3D illustrates a QRS-T scrubbed CA signal segment (in solid lines) that includes the residual R-waves 362, 364 separated by the RR interval 356, AFL waves 368-370. A blanking mask 376 is centered over the R-waves 362, 364. In connection with the R-wave 362, leading and trailing values 378, 380 of the CA signals are identified and a straight line interpolation 382 (shown in dashed lines) is drawn between the leading and trailing values 378, 380 to replace the residual R-wave 362. The process is repeated for the residual R-wave 364 to replace the residual R-wave 364 with a straight line interpolation 384 (shown in dashed lines). Once the R-waves 362, 364 are removed, the QRS-T scrubbed CA signal segment 360 exhibits a morphology that is largely driven by the flutter waves, such as AFL waves 368-370.

Returning to FIG. 3A, at 314, the one or more processors identify AFL waves in the QRS-T scrubbed CA signals. For example, the processors may identify AFL wave feature within the non-blanked portions of the QRS-T scrubbed CA sign al segment. The AFL wave features may represent peaks in the QRS-T scrubbed CA signal segment. The peaks may be identified by peak detection. Additionally or alternatively, when signal peaks are of interest, the AFL wave features may be identified by peak detection or sign changes in a derivative of the QRS-T scrubbed CA signals segment.

By way of example, the processors may detect peaks of the AFL waves utilizing the wave detection algorithm as described in patent application docket number 13246US01, Ser. No. 16/007,878, titled “METHOD AND SYSTEM TO DETECT R-WAVES IN CARDIAC ACTIVITY SIGNALS”, filed Jun. 13, 2018, the complete subject matter of which is expressly incorporated herein by reference in its entirety. The foregoing '878 application describes a computer implemented method for detecting arrhythmias in cardiac activity, comprising, among other things, under control of one or more processors configured with specific executable instructions, obtaining far field cardiac activity (CA) signals for beats; applying a direction related responsiveness (DRR) filter to the CA signals to produce DRR filtered signals; comparing a current sample from the CA signals to a prior sample from the DRR filtered signals to identify a direction characteristic of the CA signals; defining the DRR filter based on a timing constant that is set based on the direction characteristic identified; analyzing the CA signals in connection with the DRR filtered signals to identify a peak characteristic of the CA signals; determining peak to peak intervals between successive peak characteristics; and detecting at least one of noise or an arrhythmia based on the peak to peak intervals; and recording results of the detecting. While the wave detection algorithm described in foregoing '878 application is described in connection with detecting R-waves, time constants of the DRR filter may be modified to track and detect peaks in AFL waves to implement the operation at 314. By way of example, the DRR filter may be sent to have a filter coefficient utilizing a relatively short “fast up” time constant (e.g., τ=0.002 to 0.004) to respond to increases hi the incoming CA signals (e.g., during the increasing portion of an AFL wave). The DRR filter may be set to have a filter coefficient using a longer “slow down” time constant (e.g., τ=0.6 to 1.1) to respond to decreases in the incoming CA signals (e.g., during the decreasing portion of an AFL wave).

At 316, the one or more processors determine whether additional segments of the CA signals should be analyzed. If so, flow moves to 318. Otherwise, flow continues to 320.

At 318, the one or more processors increment an index to a next segment of the CA signals. Thereafter, flow returns to 310 and the operations at 310 to 316 are repeated for a next current segment of the CA signals.

Returning to 316, when the processors determine that a desired number (e.g., 10 heats, a 30 second strip of EGM signals) of QRS-T scrubbed CA signal segments have been analyzed for AFL wave features, flow moves to 320. The operations at 310-316 iteratively step through each beat of the CA signals to produce substantially fully scrubbed CA signals that do not include any QRS segments or T-wave segments.

Optionally, the operations at 310-316 may not be implemented on a beat by beat basis. Instead, the operations at 310 and/or 312 may be performed for all or a large portion of the CA signals, and thereafter the operations at 314 may identifying AFL wave features for all or the large portion of the CA signals.

Next, the operations at 320-328 analyze the blanked and scrubbed CA signals for AFL waves.

At 320, the one or more processors determine a timing or cycle length of the AFL wave features within the blanked and scrubbed CA signals. For example, when the AFL wave feature is a peak of the AFL wave, at 320, the processors identify a peak to peak time interval between successive peaks within the blanked and scrubbed CA signals as the cycle length. The processors do not calculate the AFL cycle length for peak to peak intervals that across any blanked R-wave. AFL cycle lengths that span blanked R-waves are less reliable because some AFL waves occur during the QRS interval and thus have been blanked.

Additionally or alternatively, the processors may perform a cycle-length-check for AFL waves. A cycle length check involves the processors identifying any cycle lengths that are particularly long relative to a predetermined AFL cycle length and/or an average AFL cycle length. The predetermined AFL cycle length may be preprogrammed by a clinician and/or may be automatically determined by the ICM periodically throughout operation (e.g., automatically calculate an average AFL cycle length of a past number of AFL cycle lengths). As one example, the processors may identify overly long AFL cycle lengths as AFL cycle lengths that approach double the value of a regular or average AFL cycle length. For example, an interval of 660 ms may be declared by the processors to be overly long and due to missing an intervening AFL wave. In accordance with at least one embodiment, when the processors identify a double AFL cycle length, the processors do not record the 660 interval as a single AFL cycle length. Instead, the processors either discard this long interval or record two AFL cycle lengths of 330 ms.

FIG. 3E illustrates a blanked and QRS-T scrubbed CA signal segment analyzed in accordance with an embodiment herein during the operations at 320 and 322. The blanked and QRS-T scrubbed CA signal segment includes AFL waves 393 and blanked portions centered at the R-wave markers 388. At 314, the processors identify AFL wave features 390 (also denoted by diamonds). At 320, the processors determining the AFL cycle length 392 of the AFL wave features 390, namely peak to peak intervals. At 322, the processors calculate a distribution of the AFL cycle lengths 392.

As noted above, the processors do not calculate the AFL cycle length for peak to peak intervals that include blanked R-waves as AFL cycle lengths that span blanked R-waves are less reliable. With reference to FIG. 3E, the intervals 394 represent intervals between peaks of successive AFL waves that include (or span) a blanked R-wave (as noted by the R-wave markers 388). Instead of directly calculating AFL wave intervals for the intervals 394, the processors only calculate AFL cycle lengths 392 between AFL waves 393 that occur within RR windows 396. In the foregoing manner, the process of FIG. 3A avoids calculating AFL cycle lengths for misleading “double” intervals (e.g., an interval between an AFL peak 393 on one side of a blanked R-wave and an AFL peak 393 on an opposite side of the blanked R-wave that may have one or more intervening blanked AFL peaks).

Returning to FIG. 3A, at 322, the one or more processors calculate AFL timing features based on the cycle length between AFL wave features. For example, the AFL timing features may be a distribution of the AFL cycle lengths. In accordance with one embodiment, the processors may analyze each AFL cycle length and identify within which of a series of ranges the AFL cycle length falls. For example, each range or bin may be 25 msec wide (e.g., 200-225 msec, 226-250 msec, 251-275 msec, 276-300 msec). The processors count a number of AFL events that have AFL cycle lengths within with each bin.

FIG. 3F illustrates a graphical representation of an AFL distribution 397 of AFL cycle lengths determined in accordance with an embodiment herein. The AFL distribution 397 represents a histogram that includes AFL cycle length along the horizontal axis and a count along the vertical axis. The count represents how many cardiac events exhibit a particular cycle length or a cycle length within a narrow range (e.g., within a 5.0 to 10.0 msec range). In the example of FIG. 3F, a relatively small number of AFL waves were spaced apart by an AFL cycle length of 275-285 msec or 325-350 msec, while a large number of the AFL waves were spaced apart by an AFL cycle length of 285 to 325 msec.

Returning to FIG. 3A, at 324, the one or more processors determine whether the AFL timing feature satisfies a cluster criteria. For example, the cluster criteria may correspond to a distribution of AFL cycle lengths (also referred to as AFL wave intervals). When the AFL timing feature satisfies the cluster criteria, flow moves to 326 where the processors declare an AFL episode. Alternatively, when the AFL timing feature does not satisfy the AFL cluster criteria, flow moves to 328 where the processors declare no AFL episode. By way of example, the AFL cluster criteria may be whether the AFL distribution exhibits a select (e.g., dominating) peak within a select timing range (e.g., near 300 msec, between 275-325 msec, etc.). The AFL cluster criteria may represent an indication of whether a histogram of the cycle length distribution represents an AFL episode. For instance, an AFL episode will exhibit a distribution that is narrow with a very small standard deviation as the AFL cycle length remains stable beat to beat. Accordingly, in accordance with at least one embodiment, at 322, the one or more processors generate a histogram that maintains a count of AFL cycle lengths within predetermined bins. At 324, the processors determine whether the count within a select one or more of the predetermined bins exceeds a threshold. Optionally, the processors may use a coefficient of variation (standard deviation/mean) to determine the regularity of the AFL waves.

Additionally, or alternatively, with reference to FIG. 3F, the processors may compare (at 324) the counts of AFL cycle lengths within select cycle length ranges to one or more distribution thresholds (representing a cluster criteria). For example, when the count of AFL cycle lengths within one or more bins in a cycle length range of 250-353 msec exceeds a count threshold, the processors declare an AFL episode. As another example, the distribution threshold may be characterizes by a ratio or relation of counts of cycle lengths in adjacent bins. For example, when a select majority (e.g., 70% or more) the cycle lengths counted over a broader range (e.g., 250-350 msec) fall within a narrow range (e.g., 275-325 msec), the processors may determine that the AFL timing feature satisfies the cluster criteria.

FIG. 4 illustrates a process for building a QRS-T template in accordance with embodiments herein. The operations of FIG. 4 may be performed by one or more processors of an ICM, a local external device and/or a remote server. The operations of FIG. 4 may be performed contemporaneous with, or at a point in time prior to, the operations of FIG. 3A. For example, the operations of FIG. 4 may be performed by the ICM in real-time in connection with the operations of FIG. 3A. Additionally or alternatively, the operations of FIG. 4 may be performed at a time of implant, periodically and/or during a patient-clinic visit to build one or more QRS-T templates indicative of the patient's QRS-T complex. The QRS-T template(s) may be built by a local external device and/or a remote server based on collections of CA signals that are wirelessly transmitted from the ICM. The local external device and/or remote server may then transmit the QRS-T template(s) back to the ICM for use in connection with the operations of FIG. 3A.

At 402, the one or more processors obtain a CA data set (CA signals and device document markers) and step an index along the CA signals to a current device document R-wave marker.

At 404, the one or more processors overlay a template collection window onto a current beat in the CA signals, wherein the template collection window centered or otherwise positioned relative to, the current R-wave marker.

At 406, the one or more processors record the QRS-T complex from the current beat of the CA signals within the template collection window.

At 408, the one or more processors determine whether a sufficient number of QRS-T complexes have been collected. For example, it may be desirable to collect a certain number of QRS-T complexes to build each individual template. Accordingly, the processors may determine when a desired number of QRS-T complexes have been collected. When additional QRS-T complexes are to be collected, flow moves to 410. At 410, the one or more processors increment the index into the CA signals to the next device documented R-wave marker. Alternatively, at 408, when a desired number of QRS-T complexes have been collected, flow moves to 412.

At 412, the one or more processors build the QRS-T template from the collection of QRS-T complexes. The QRS-T template is built by combining segments of the CA signals surrounding device documented R-wave markers. For example, a segment beginning 300 ms before and continuing 400 ms after each R-wave marker may be collected from all or a select portion of the detected beats. For example, the QRS-T template may be built as an ensemble average of the QRS-T complexes. To calculate the ensemble average, the QRS-T complexes may be aligned, such as based on the starting and ending points of each QRS-T complex, based on peaks of the R-waves and/or based on peaks of the T-waves therein. The QRS-T template will exhibit a morphology that generally corresponds to an R-wave and a T-wave for the patient, but will not exhibit a particular morphology for an AFL wave. Because R-T interval is very stable, all the T-waves occur at the same distance/time from R-wave. On the other hand, the AFL waves occur at different time of the cardiac cycle. The ensemble average does not include a flutter wave component as flutter waves occur at different times in a cardiac cycle and thus are generally not temporally aligned with the R-wave (or a device documented R-wave marker). Accordingly, the ensemble averaging process tends to cancel out the flutter wave components.

The one or more processors may utilize various R-wave detection algorithms to detect R-wave peaks and utilize the R-wave peaks to align the QRS-T complexes with one another. By way of example, the processors may utilize an R-wave detection algorithm as described in '878 application. The R-waves and T-waves within the QRS-T complexes are averaged using a longest interval available from the collection of QRS-T complexes based on the shortest RR interval.

Additionally, or alternatively, segments of the CA signals that correspond to individual beats may be removed/omitted from the QRS-T template building operation based on certain criteria. For example, individual beats may be removed/omitted based on an RR interval analysis and/or a QRS morphology matching analysis. For example, the one or more processors may analyze RR intervals between one or more beats and determine that the RR intervals differ sufficiently from an expected RR interval to justify removing the corresponding beat from the QRS-T template. Additionally, or alternatively, the one or more processors may analyze a QRS morphology of each beat (e.g., relative to a baseline QRS morphology). Based on a relation between a current QRS morphology and a baseline QRS morphology, the processors may determine whether to include an individual beat within the QRS-T template. In certain instances, it may be desirable to apply an RR interval analysis and/or QRS morphology matching as a threshold to include beats within the QRS-T template in order to avoid including beats that were inappropriately sensed, beats that correspond to premature ventricular contractions and the like.

In accordance with various embodiments, different QRS-T templates may be generated and stored. By way of example, a first QRS-T template may be generated in connection with a first range of heart rates, while a different second QRS-T template is generated in connection with a second range of heart rates. During operation of the process of FIG. 3A, an appropriate QRS-T template is selected based on the present heart rate of the patient, Additionally, or alternatively, the process of FIG. 4 may generate QRS-Templates and a separate T-wave templates. During the operations of FIG. 3A, a desired QRS-Template and a desired T-wave template may be selected based on various criteria.

The foregoing description is provided in connection with operating upon far field CA signals, Additionally or alternatively, the foregoing operations may be implemented in connection with near field CA signals.

One or more embodiments here are described in connection with an ICM type of implantable medical device. Optionally, embodiments may be implemented in connection with one or more other types of implantable medical devices (IMDs). Non-limiting examples of IMDs include one or more of neurostimulator devices, implantable leadless monitoring and/or therapy devices, and/or alternative implantable medical devices. For example, the IMD may represent a cardiac monitoring device, pacemaker, cardioverter, cardiac rhythm management device, defibrillator, neurostimulator, leadless monitoring device, leadless pacemaker and the like. For example, the IMD may include one or more structural and/or functional aspects of the device(s) described in U.S. Pat. No. 9,333,351 “Neurostimulation Method and System to Treat Apnea” and U.S. Pat. No. 9,044,610 “System And Methods For Providing A Distributed Virtual Stimulation Cathode For Use With An Implantable Neurostimulation System”, which are hereby incorporated by reference. Additionally or alternatively, the IMD may include one or more structural and/or functional aspects of the device(s) described in U.S. Pat. No. 9,216,285 “Leadless Implantable Medical Device Having Removable and Fixed Components” and U.S. Pat. No. 8,831,747 “Leadless Neurostimulation Device And Method Including The Same”, which are hereby incorporated by reference. Additionally or alternatively, the IMD may include one or more structural and/or functional aspects of the device(s) described in U.S. Pat. No. 8,391,980 “Method and System for Identifying a Potential Lead Failure in an Implantable Medical Device” and U.S. Pat. No. 9,232,485 “System And Method For Selectively Communicating With An Implantable Medical Device”, which are hereby incorporated by reference.

### Closing

The various methods as illustrated in the Figures and described herein represent exemplary embodiments of methods. The methods may be implemented in software, hardware, or a combination thereof. In various of the methods, the order of the steps may be changed, and various elements may be added, reordered, combined, omitted, modified, etc. Various of the steps may be performed automatically (e.g., without being directly prompted by user input) and/or programmatically (e.g., according to program instructions).

Various modifications and changes may be made as would be obvious to a person skilled in the art having the benefit of this disclosure. It is intended to embrace all such modifications and changes and, accordingly, the above description is to be regarded in an illustrative rather than a restrictive sense.

Various embodiments of the present disclosure utilize at least one network that would be familiar to those skilled in the art for supporting communications using any of a variety of commercially-available protocols, such as Transmission Control Protocol/Internet Protocol (“TCP/IP”), User Datagram Protocol (“UDP”), protocols operating in various layers of the Open System Interconnection (“OSI”) model, File Transfer Protocol (“FTP”), Universal Plug and Play (“UpnP”), Network File System (“IFS”), Common Internet File System (“CIFS”) and AppleTalk. The network can be, for example, a local area network, a wide-area network, a virtual private network, the Internet, an intranet, an extranet, a public switched telephone network, an infrared network, a wireless network, a satellite network and any combination thereof.

In embodiments utilizing a web server, the web server can run any of a variety of server or mid-tier applications, including Hypertext Transfer Protocol (“HTTP”) servers, FTP servers, Common Gateway Interface (“CGI”) servers, data servers, Java servers, Apache servers and business application servers. The server(s) also may be capable of executing programs or scripts in response to requests from user devices, such as by executing one or more web applications that may be implemented as one or more scripts or programs written in any programming language; such as Java®, C, C# or C++, or any scripting language, such as Ruby, PHP, Perl, Python or TCL, as well as combinations thereof. The server(s) may also include database servers; including without limitation those commercially available from Oracle®, Microsoft®, Sybase® and IBM® as well as open-source servers such as MySQL, Postgres, SQLite, MongoDB, and any other server capable of storing, retrieving and accessing structured or unstructured data. Database servers may include table-based servers, document-based servers, unstructured servers; relational servers, non-relational servers or combinations of these and/or other database servers.

The environment can include a variety of data stores and other memory and storage media as discussed above. These can reside in a variety of locations, such as on a storage medium local to (and/or resident in) one or more of the computers or remote from any or all of the computers across the network. In a particular set of embodiments, the information may reside in a storage-area network (“SAN”) familiar to those skilled in the art. Similarly, any necessary files for performing the functions attributed to the computers, servers or other network devices may be stored locally and/or remotely, as appropriate. Where a system includes computerized devices, each such device can include hardware elements that may be electrically coupled via a bus, the elements including, for example, at least one central processing unit (“CPU” or “processor”), at least one input device (e.g., a mouse, keyboard, controller, touch screen or keypad) and at least one output device (e.g., a display device, printer or speaker). Such a system may also include one or more storage devices, such as disk drives, optical storage devices and solid-state storage devices such as random access memory (“RAM”) or read-only memory (“ROM”), as well as removable media devices, memory cards, flash cards, etc.

Such devices also can include a computer-readable storage media reader, a communications device (e.g., a modem, a network card (wireless or wired), an infrared communication device, etc.) and working memory as described above. The computer-readable storage media reader can be connected with, or configured to receive, a computer-readable storage medium, representing remote, local, fixed and/or removable storage devices as well as storage media for temporarily and/or more permanently containing, storing, transmitting and retrieving computer-readable information. The system and various devices also typically will include a number of software applications, modules, services or other elements located within at least one working memory device, including an operating system and application programs, such as a client application or web browser. It should be appreciated that alternate embodiments may have numerous variations from that described above. For example, customized hardware might also be used and/or particular elements might be implemented in hardware, software (including portable software, such as applets) or both. Further, connection to other computing devices such as network input/output devices may be employed.

Various embodiments may further include receiving, sending, or storing instructions and/or data implemented in accordance with the foregoing description upon a computer-readable medium. Storage media and computer readable media for containing code, or portions of code, can include any appropriate media known or used in the art, including storage media and communication media, such as, but not limited to, volatile and non-volatile, removable and non-removable media implemented in any method or technology for storage and/or transmission of information such as computer readable instructions, data structures, program modules or other data, including RAM, ROM, Electrically Erasable Programmable Read-Only Memory (“EEPROM”), flash memory or other memory technology, Compact Disc Read-Only Memory (“CD-ROM”), digital versatile disk (DVD) or other optical storage, magnetic cassettes, magnetic tape, magnetic disk storage or other magnetic storage devices or any other medium which can be used to store the desired information and which can be accessed by the system device. Based on the disclosure and teachings provided herein, a person of ordinary skill in the art will appreciate other ways and/or methods to implement the various embodiments.

The specification and drawings are, accordingly, to be regarded n an illustrative rather than a restrictive sense. It will, however, be evident that various modifications and changes may be made thereunto without departing from the broader spirit and scope of the invention as set forth in the claims.

Other variations are within the spirit of the present disclosure. Thus, while the disclosed techniques are susceptible to various modifications and alternative constructions, certain illustrated embodiments thereof are shown in the drawings and have been described above in detail. It should be understood, however, that there is no intention to limit the invention to the specific form or forms disclosed, but on the contrary, the intention is to cover all modifications, alternative constructions and equivalents falling within the spirit and scope of the invention, as defined in the appended claims.

The use of the terms “a” and “an” and “the” and similar referents in the context of describing the disclosed embodiments (especially in the context of the following claims) are to be construed to cover both the singular and the plural, unless otherwise indicated herein or clearly contradicted by context. The terms “comprising,” “having,” “including” and “containing” are to be construed as open-ended terms (i.e., meaning “including, but not limited to,”) unless otherwise noted. The term “connected,” when unmodified and referring to physical connections, is to be construed as partly or wholly contained within, attached to or joined together, even if there is something intervening. Recitation of ranges of values herein are merely intended to serve as a shorthand method of referring individually to each separate value falling within the range, unless otherwise indicated herein and each separate value is incorporated into the specification as if it were individually recited herein. The use of the term “set” (e.g., “a set of items”) or “subset” unless otherwise noted or contradicted by context, is to be construed as a nonempty collection comprising one or more members. Further, unless otherwise noted or contradicted by context, the term “subset” of a corresponding set does not necessarily denote a proper subset of the corresponding set, but the subset and the corresponding set may be equal.

Operations of processes described herein can be performed in any suitable order unless otherwise indicated herein or otherwise clearly contradicted by context. Processes described herein (or variations and/or combinations thereof) may be performed under the control of one or more computer systems configured with executable instructions and may be implemented as code (e.g., executable instructions, one or more computer programs or one or more applications) executing collectively on one or more processors, by hardware or combinations thereof. The code may be stored on a computer-readable storage medium, for example, in the form of a computer program comprising a plurality of instructions executable by one or more processors. The computer-readable storage medium may be non-transitory.

All references, including publications, patent applications and patents, cited herein are hereby incorporated by reference to the same extent as if each reference were individually and specifically indicated to be incorporated by reference and were set forth in its entirety herein.

It is to be understood that the subject matter described herein is not limited in its application to the details of construction and the arrangement of components set forth in the description herein or illustrated in the drawings hereof. The subject matter described herein is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the invention without departing from its scope. While the dimensions, types of materials and physical characteristics described herein are intended to define the parameters of the invention, they are by no means limiting and are exemplary embodiments. Many other embodiments will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means—plus-function format and are not intended to be interpreted based on 35 U.S.C. § 112(f), unless and until such claim limitations expressly use the phrase “means for” followed by a statement of function void of further structure.

What is claimed is:

1. A computer implemented method for detecting arrhythmias in cardiac activity, comprising:
under control of one or more processors configured with specific executable instructions,
obtaining cardiac activity (CA) signals for a series of beats;
building a QRS-T template based on an ensemble of QRS complexes within the CA signals;
subtracting the QRS-T template from the CA signals to obtain QRS-T scrubbed CA signals;
determining an atrial flutter (AFL) timing feature within the QRS scrubbed CA signals; and
declaring an AFL episode based on a relation between the AFL timing feature and an AFL cluster criteria.

2. The method of claim 1, further comprising determining cycle lengths of AFL wave features within the QRS scrubbed CA signals, the AFL timing feature determined based on the cycle lengths.

3. The method of claim 2, wherein the AFL timing feature represents a distribution of the cycle lengths.

4. The method of claim 2, wherein the declaring operation comprises declaring the AFL episode when the AFL timing feature exhibits a distribution of cycle lengths having a select peak within a select timing range.

5. The method of claim 1, further comprising, before performing the subtracting operation, aligning the QRS-T template with each QRS-T segment in the CA signals.

6. The method of claim 5, further comprising scaling the QRS-T template based on the amplitude of each QRS-T segment in the CA signals before the subtracting operation.

7. The method of claim 1, wherein the building operation comprises building a QRS-T template and wherein the subtracting operation comprises subtracting the QRS-T template from each QRS-T segment in the CA signals to calculate the QRS-T scrubbed CA signals.

8. The method of claim 1, wherein the QRS-T scrubbed CA signals include residual QRS complex, the method further comprising applying a blanking mask to blank a QRS segment of the QRS-T scrubbed CA signal to eliminate a residual QRS components from the QRS-T scrubbed CA signals.

9. The method of claim 8, wherein the applying the blanking mask comprises identifying leading and trailing values of the CA signals at the beginning and ending points of the blanking mask and replacing the CA signals with a straight-line signal between the beginning and ending points.

10. The method of claim 1, further comprising generating a histogram that maintains a count of AFL cycle lengths within predetermined bins, the determining operation determining whether the count within a select one or more of the predetermined bins exceeds a threshold.

11. A system for detecting arrhythmias in cardiac activity, comprising:
memory to store specific executable instructions;
one or more processors configured to execute the specific executable instructions for:
obtaining cardiac activity (CA) signals for a series of beats;
building a QRS-T template based on an ensemble of QRS complexes within the CA signals;
subtracting the QRS-T template from the CA signals to obtain QRS scrubbed CA signals;
determining an atrial flutter (AFL) timing feature within the QRS scrubbed CA signals; and
declaring an AFL episode based on a relation between the AFL timing feature and an AFL cluster criteria.

12. The system of claim 11, wherein the one or more processors are further configured to determine cycle lengths of AFL wave features within the QRS scrubbed CA signals, the AFL timing feature determined based on the cycle lengths.

13. The system of claim 12, wherein the AFL timing feature represents a distribution of the cycle lengths.

14. The system of claim 11, wherein the one or more processors are configured to declare the AFL episode when the AFL timing feature exhibits a distribution of cycle lengths having a select peak within a select timing range.

15. The system of claim 11, wherein the one or more processors are further configured to align the QRS-T template with an R-wave marker of a QRS complex of the CA signals before performing the subtracting operation.

16. The system of claim 15, wherein the one or more processors are further configured to scale the QRS-Template based on the amplitude of QRS-T segment in the CA signals before the subtracting operation.

17. The system of claim 11, wherein the building operation comprises building, as the QRS-Template, a QRS-T template and wherein the subtracting operation comprises subtracting the QRS-T template from the CA signals to calculate the QRS-T scrubbed CA signals.

18. The system of claim 11, wherein the QRS scrubbed CA signals include residual QRS complex, and wherein the one or more processors are configured to apply a blanking mask to blank a QRS segment of the QRS-T scrubbed CA signal to eliminate a residual QRS components from the QRS-T scrubbed CA signals.

19. The system of claim 11, further comprising an implantable medical device housing the processor and memory.

20. The system of claim 11, wherein the processor and memory are housed within at least one of a local external device and a remote server.
